(19) United States
(12) Patent Application Publication
(10) Pub. No.: US 2022/0032027 A1
(43) Pub. Date: Feb. 3, 2022

(54) HOLLOW MICRONEEDLE FOR TRANSDERMAL DELIVERY OF ACTIVE MOLECULES AND/OR FOR THE SAMPLING OF BIOLOGICAL FLUIDS AND MANUFACTURING METHOD OF SUCH HOLLOW MICRONEEDLE

(71) Applicant: Altergon SA, Lugano (CH)

(72) Inventors: Luca DE STEFANO, Napoli (IT); Principia DARDANO, Ercolano, Naples (IT); Luigi NICOLAIS, Ercolano, Naples (IT)

(73) Assignee: Altergon SA, Lugano (CH)

(21) Appl. No.: 17/275,724

(22) PCT Filed: May 21, 2019
(86) PCT No.: PCT/IB2019/054168

§ 371 Date: Mar. 12, 2021

(30) Foreign Application Priority Data
Jun. 20, 2018 (IT) 102018000006526

(51) Int. Cl. A61M 37/00

(52) U.S. Cl. CPC A61M 37/0015; A61M 2037/0007; A61M 2037/0023; G03F 7/20; A61M 2037/0053; A61M 2205/368; A61M 2037/0061

(57) ABSTRACT

The present invention relates to a micro-needle (7; 8; 9) for the transdermal administration of active molecules and/or for the sampling of biological fluids. The micro-needle (7; 8; 9) is made of polymeric material through photolithography. A cavity is defined in the micro-needle (7; 8; 9).

The present invention further relates to a method for obtaining through photolithography at least one micro-needle (7; 8; 9) for the transdermal administration of active molecules and/or for the sampling of biological fluids. A photo-cross linking polymer is exposed in liquid phase to an energy radiation causing the hardening thereof. A photolithographic mask (1; 2) is interposed between the source of the energy radiation and the photo-cross linking polymer. The photolithographic mask (1; 2) is configured in a manner such to generate in the photo-cross linking polymer a peripheral shadow area, a central shadow area and a lighting area confined between the peripheral shadow area and the central shadow area.

The method according to this invention is aimed at obtaining a micro-needle (7; 8; 9) for the transdermal administration of active molecules and/or for the sampling of biological fluids which shows the peculiar characteristic of being hollow and which is manufactured by means of a single photolithography operation, thus avoiding the use of additional processing.

The present invention relates to a method for obtaining by photolithography at least one micro-needle for the transdermal administration of active molecules and/or for the sampling of biological fluids, a micro-needle obtained by this method and a device for the transdermal administration of active molecules and/or for the sampling of biological fluids comprising such a micro-needle.

The present invention relates to the manufacture of biomedical devices for sampling biological fluids, such as sweat, lymph, blood, and the controlled release of active molecules, such as drugs or vaccines, both for topical use and for systemic use. The sampling of biological fluids for diagnostic analysis, as well as the transdermal administration of drugs, by means of devices that use needles is often problematic both for the fear of pain and, in some subjects, also of the needles themselves (belonephobia). Moreover, for some pathologies subject to a possible wide diffusion, such as exanthematous diseases and in general all those involving large-scale compulsory vaccination programs, as well as those requiring to be monitored daily or several times a day, the use of syringes with standard needles can become an invasive practice, in some cases hardly tolerable. On the other hand, the transdermal administration of drugs is not very effective considering that the skin is a multilayer tissue, a natural barrier against agents that are external to the human body. In recent years, to overcome these limitations, various technological solutions have been presented both with regard to materials and devices to be used. In particular, the possibility of micro-processing materials, both organic and inorganic, has consented the fabrication of devices based on micro-needles of variable length, from a millimeter to a few hundred micrometers, with mechanical properties such as the possibility to be introduced in the first layers of the dermis without reaching the layer affected by the presence of the nerves and thus completely eliminating the sensation of pain linked to the penetration of the needles.

Several examples of devices for the transdermal administration of active molecules and/or for the sampling of biological fluids comprising a plurality of micro-needles are known to date, such examples of devices being made of inorganic materials (silicon, glass, mixed oxides) or organic materials (polymers, plastics, celluloses). All the manufacturing methods presented involve a combination of more or less complex technological processes, easily achievable and controllable on a laboratory scale, but difficult to implement on a large industrial scale. In fact this feature prevents the economic and technological feasibility of a production on industrial scale of this type, so that the presence of commercial devices on the market is extremely small.

The document US2013/0150822A1 relates to a technical solution for increasing the permeability of drugs in the skin by means of a device comprising nano-structures arranged in a predetermined pattern on the side of the device intended to come into contact with the patient's skin. The device is made in the form of a transdermal patch that includes a reservoir where the drug is loaded; a membrane that acts as a control membrane, slowing down the rate of drug release; a removable layer which inhibits the release of the drug until the removal of this layer and a plurality of micro-needles which penetrate the patient's skin. In the document US2013/0150822A1, the nano-imprinting process is used to obtain the micro-needles on the device. This process involves the use of lithography only to obtain the master defining the planar geometry of the micro-needles (in particular diameter and distance), after which the replicas are obtained by molding. Following the molding of the replicas, the channels are first etched in the micro-needles and then filled to obtain suitable permeability.

The document CN107297020A concerns the manufacture of hollow micro-needles through different steps, including a step of metal deposition for electroplating. A photolithographic step is required to obtain a sacrificial layer, later removed. The document EP3300765A1 describes an array of hollow micro-needles, which are manufactured by molding and then subjected to drilling. A further array of hollow micro-needles is described in the document CN102530848A, wherein the fabrication takes place by anisotropic etching (chemical in KOH) of the silicon. In the document CN106176573A, the micro-needles are manufactured by depositing and centrifuging hyaluronic acid around one mold and using a sacrificial layer. Finally, the document US2015/0335871A1 describes a method of manufacturing micro-needles in which equipment is used according to Electro-Discharge Machining (EDM) technology or according to Computerized Numerical Controlled (CNC) technology to cut metal needles from metal blocks. The internal channel of the hollow micro-needles is obtained by adding a further drilling step.

The document US2006/0015061A1 discloses a device for the transdermal administration of active molecules and/or for the sampling of biological fluids comprising an array of hollow micro-needles, having a monolithic structure according to which the micro-needles extend perpendicularly starting from a support substrate. The device is obtained by adopting a technique of partial photolithography, wherein the photolithography is only used to obtain the master defining the geometry of the micro-needles, afterwards the replicas are obtained starting from this master. A mask, whose inner and outer profiles are concentric to each other, is used for the photolithography of the master. A concave sacrificial layer is then adopted in order to shape the apical portions of the master, thus obtaining inclined ends of the micro-needles. The document US2014/0124898A1 discloses microstructures or nano-structures that can be used as elements for high-tech batteries.

A first objective of the invention is to allow a simple and fast production of micro-needles, in particular of hollow micro-needles, usable for the transdermal administration of active molecules and/or for the sampling of biological fluids, the manufacture of micro-needles taking place through a number of extremely reduced phases if compared to the manufacturing methods known to the current state of art.

A second objective of the invention is to allow the production of micro-needles, in particular of hollow micro-needles, which can be used for the transdermal administration of active molecules and/or for the sampling of biological fluids, by means of a cost-effective process designed for large-scale industrial implementation.

A third objective of the invention is to provide a device whose production method allows to make easy and quick changes to the shape, length and mechanical properties of the micro-needles.

A fourth objective of the invention is to provide a device for the transdermal administration of active molecules and/or for the sampling of biological fluids, which is biocompatible in a manner such that, when in contact with the skin, it does not cause irritation or infections, and it is solid and flexible enough to adapt to any point of application on the human body.

A fifth objective of the invention is to provide a device for the transdermal administration of active molecules and/or for the sampling of biological fluids, characterized by considerable versatility and therefore suitable to be used in multiple therapeutic and diagnostic applications, for cosmetic or biomedical use.

A sixth objective of the invention is to provide a production method for a device for the transdermal administration of active molecules and/or for the sampling of biological fluids, capable of ensuring optimal repeatability and optimal precision, with very small tolerances in the dimensions of the components.

A seventh objective of the invention is to provide a device for the transdermal administration of active molecules, which is effectively adaptable to the specificities of each drug or vaccine with regard to the dosage, release time and release mode.

An eighth objective of the invention is to provide a device for the transdermal administration of active molecules, which can be integrated into control networks and which can interface with electronic control devices.

A ninth objective of the invention is to provide a device for the transdermal administration of active molecules, prepared for operation modes that involve a release of the active molecules, which is actively adjustable and/or controllable.

All the objectives are fully achieved by the present invention, which includes the aspects listed below.

A first independent aspect of the invention relates to a method for manufacturing by photolithography at least one micro-needle for the transdermal administration of active molecules and/or for the sampling of biological fluids, comprising the step of:
exposing a photo-cross linking polymer in liquid phase to an energy radiation causing the hardening thereof, a photolithographic mask being interposed between the source of said energy radiation and said photo-cross linking polymer, said photolithographic mask being configured in a manner such to generate in said photo-cross linking polymer a peripheral shadow area, a central shadow area and a lighting area confined between said peripheral shadow area and said central shadow area, specifically with the aim to obtain a hollow micro-needle by means of said photolithography.

A second aspect of the invention, depending on the first aspect, relates to a method for obtaining by photolithography at least one micro-needle for the transdermal administration of active molecules and/or for sampling biological fluids, wherein said photolithographic mask comprises a peripheral region of impermeability to said energy radiation and a central region of impermeability to said energy radiation, said peripheral region being suitable to generate said peripheral shadow area and said central region being suitable to generate said central shadow area, and wherein said peripheral region and said central region are distinct and separate from each other.

A third aspect of the invention, depending on the second aspect, relates to a method for obtaining by photolithography at least one micro-needle for the transdermal administration of active molecules and/or for sampling of biological fluids, wherein, the outer profile of said photolithographic mask being the line which internally delimits said peripheral region and the inner profile of said photolithographic mask being the line that externally delimits said central region, said outer profile entirely encloses said inner profile.

A fourth aspect of the invention, depending on the third aspect, relates to a method for obtaining by photolithography at least one micro-needle for the transdermal administration of active molecules and/or for the sampling of biological fluids, wherein said outer profile and said inner profile are circular or elliptical or polygonal profiles.

A fifth aspect of the invention, depending on the third aspect or on the fourth aspect, relates to a method for obtaining by photolithography at least one micro-needle for the transdermal administration of active molecules and/or for sampling of biological fluids, wherein the characteristic dimension, in particular the diameter or the diagonal, of said outer profile is comprised between 100 micrometers and 910 micrometers, preferably between 300 micrometers and 900 micrometers, even more preferably about 500 micrometers and/or wherein the characteristic dimension, in particular the diameter or diagonal, of said inner profile is between 90 micrometers and 900 micrometers, preferably between 100 micrometers and 700 micrometers, even more preferably about 300 micrometers and/or wherein the distance between said outer profile and said inner profile is between 10 micrometers and 200 micrometers, preferably between 60 micrometers and 140 micrometers, even more preferably and about 100 micrometers.

A sixth aspect of the invention, depending on any one of the aspects from the third aspect to the fifth aspect, relates to a method for obtaining by photolithography at least one micro-needle for the transdermal administration of active molecules and/or for sampling biological fluids, wherein the geometric center of said outer profile is substantially coincident with the geometric center of said inner profile, specifically with the aim of obtaining a substantially symmetrical extension of said micro-needle during said photolithography.

A seventh aspect of the invention, depending on any of the aspects from the third aspect to the fifth aspect, relates to a method for obtaining by photolithography at least one micro-needle for the transdermal administration of active molecules and/or for sampling of biological fluids, wherein the geometric center of said outer profile is arranged at a predetermined distance with respect to the geometric center of said inner profile, specifically with the aim of obtaining an asymmetrical extension of said micro-needle during said photolithography.

An eighth aspect of the invention, depending on the seventh aspect, relates to a method for obtaining by photolithography at least one micro-needle for the transdermal administration of active molecules and/or for sampling biological fluids, wherein the predetermined distance between the geometric center of said external profile and the geometric center of said inner profile is comprised between 10 micrometers and 200 micrometers, preferably between 30 micrometers and 50 micrometers, even more preferably about 40 micrometers.

A ninth aspect of the invention, depending on any of the aspects from the first aspect to the eighth aspect, relates to a method for obtaining by photolithography at least one micro-needle for the transdermal administration of active molecules and/or for sampling biological fluids, further comprising the step of:
interrupting the exposure of said photo-cross linking polymer to said energy radiation before a predetermined duration, specifically with the aim of obtaining a cavity passing through said micro-needle.

A tenth aspect of the invention, depending on any of the aspects from the first aspect to the eighth aspect, relates to a method for obtaining by photolithography at least one micro-needle for the transdermal administration of active molecules and/or for sampling biological fluids, further comprising the step of:
interrupting the exposure of said photo-cross polymer to said energy radiation after a predetermined duration, specifically with the aim of obtaining a blind cavity in said micro-needle.

An eleventh aspect of the invention, depending on any of the aspects from the first aspect to the eighth aspect, relates to a method for obtaining by photolithography at least one micro-needle for transdermal administration of active molecules and/or for sampling biological fluids, further comprising the step of:
setting the power of said source of said energy radiation below a predetermined power, specifically with the aim of obtaining a through cavity in said micro-needle.

A twelfth aspect of the invention, depending on any of the aspects from the first aspect to the eighth aspect, relates to a method for obtaining by photolithography at least one micro-needle for the transdermal administration of active molecules and/or for sampling biological fluids, further comprising the step of:
setting the power of said source of said energy radiation above a predetermined power, specifically with the aim of obtaining a blind cavity in said micro-needle.

A thirteenth aspect of the invention, depending on any of the aspects from the first aspect to the twelfth aspect, relates to a method for obtaining by photolithography at least one micro-needle for the transdermal administration of active molecules and/or for sampling of biological fluids, wherein said energy radiation is an ultraviolet radiation.

A fourteenth aspect of the invention, depending on any of the aspects from the first aspect to the thirteenth aspect, relates to a method for obtaining by photolithography at least one micro-needle for the transdermal administration of active molecules and/or for sampling biological fluids, wherein said photo-cross polymer is polyethylene glycol (PEG).

A fifteenth aspect of the invention, depending on any of the aspects from the first aspect to the fourteenth aspect, relates to a method for obtaining by photolithography at least one micro-needle for the transdermal administration of active molecules and/or for the sampling of biological fluids, wherein said photo-cross linking polymer is added with a photoinitiator, in particular Darocur or Irgacure or LAP.

A sixteenth aspect of the invention, depending on any of the aspects from the first aspect to the fifteenth aspect, relates to a method for obtaining by photolithography at least one micro-needle for the transdermal administration of active molecules and/or for sampling biological fluids, wherein the said photo-cross polymer is added with a photosensitive polymer or with a photosensitive compound, specifically with the aim of making said micro-needle suitable to be used for releasing an active ingredient only upon exposing said micro-needle to a coherent radiation with a predetermined wavelenght.

A seventeenth aspect of the invention, depending on any of the aspects from the first aspect to the sixteenth aspect, relates to a method for obtaining by photolithography at least one micro-needle for the transdermal administration of active molecules and/or for sampling biological fluids, wherein said photo-cross polymer is added with metal particles, preferably with particles of a noble metal, even more preferably with gold particles, specifically with the aim of making said micro-needle suitable for releasing an active ingredient only upon heating said micro-needle by radiation.

An eighteenth aspect of the invention, depending on any of the aspects from the first aspect to the seventeenth aspect, relates to a method for obtaining by photolithography at least one micro-needle for the transdermal administration of active molecules and/or sampling biological fluids, wherein said photo-cross polymer is added with an active ingredient.

A nineteenth aspect of the invention, depending on any of the aspects from the first aspect to the eighteenth aspect, relates to a method for obtaining by photolithography at least one micro-needle for the transdermal administration of active molecules and/or for sampling biological fluids, wherein the molecular weight of said photo-cross polymer can be modular so as to confer to said micro-needle morphological characteristics such to adjust the speed of release of the molecules of an active ingredient through said micro-needle.

A twentieth aspect of the invention, depending on any of the aspects from the first aspect to the nineteenth aspect, relates to a method for obtaining by photolithography at least one micro-needle for the transdermal administration of active molecules and/or for sampling biological fluids, wherein the wettability of said photo-cross polymer can be modular so as to confer to said micro-needle surface chemical characteristics and/or according to the hydrophobic and/or hydrophilic nature of the molecules of an active ingredient to be released through said micro-needle.

A twenty-first aspect of the invention, depending on any of the aspects from the first aspect to the twentieth aspect, relates to a method for obtaining by photolithography at least one micro-needle for the transdermal administration of active molecules and/or for sampling biological fluids, wherein said micro-needle is generated on a surface of a support element, said surface of said support element having an opening at the position intended for said micro-needle.

A twenty-second aspect of the invention, depending on the twenty-first aspect, relates to a method for obtaining by photolithography at least one micro-needle for the transdermal administration of active molecules and/or for sampling biological fluids, wherein said photo-cross polymer is stored in a container, preferably made of silicone, and said support element is placed on said container so as to be at direct contact with said photo-cross linking polymer.

A twenty-third aspect of the invention, depending on any of the aspects from the first aspect to the twenty-second aspect, relates to a method for obtaining by photolithography at least one micro-needle for the transdermal administration of active molecules and/or for sampling biological fluids, further comprising the step of:
removing from said micro-needle the non-hardened photo-cross linking polymer by washing said micro-needle, in particular in deionized water.

A twenty-fourth independent aspect of the invention, dependent on any of the aspects from the first aspect to the twenty-third aspect, relates to a method for obtaining by photolithography at least one micro-needle for the transdermal administration of active molecules and/or for sampling biological fluids, wherein said micro-needle is obtained simultaneously with at least one further micro-needle, optionally simultaneously with a plurality of micro-needles positioned according to a predetermined regular and/or orderly arrangement.

A twenty-fifth independent aspect of the invention relates to a micro-needle for the transdermal administration of active molecules and/or for sampling of biological fluids, said micro-needle being made of polymeric material by photolithography, wherein a cavity is defined in said micro-needle.

A twenty-sixth aspect of the invention, depending on the twenty-fifth aspect, relates to a micro-needle for the transdermal administration of active molecules and/or for sampling biological fluids, wherein said micro-needle is straight truncated-cone shaped or regular truncated-pyramid shaped and wherein said cavity is a through cavity.

A twenty-seventh aspect of the invention, depending on the twenty-fifth aspect, relates to a micro-needle for the transdermal administration of active molecules and/or for sampling biological fluids, wherein said micro-needle is truncated cone shaped or regular pyramid shaped and wherein said cavity is a blind cavity.

A twenty-eighth aspect of the invention, depending on the twenty-fifth aspect, relates to a micro-needle for the transdermal administration of active molecules and/or for sampling biological fluids, wherein said micro-needle is oblique truncated cone shaped or irregular truncated pyramid shaped and wherein said cavity is a through cavity.

A twenty-ninth aspect of the invention, depending on any of the aspects from the twenty-fifth aspect to the twenty-eighth aspect, relates to a micro-needle for the transdermal administration of active molecules and/or for sampling biological fluids, wherein the height of said micro-needle is comprised between 200 micrometers and 2000 micrometers, preferably between 900 micrometers and 1300 micrometers, even more preferably about 1100 micrometers, and/or wherein the base of said micro-needle has a diameter or diagonal with extension comprised between 100 micrometers and 900 micrometers, preferably between 300 micrometers and 700 micrometers, even more preferably about 500 micrometers, and/or wherein the thickness of the walls of said micro-needle is between 10 micrometers and 200 micrometers, preferably between 60 micrometers and 140 micrometers, even more preferably about 100 micrometers.

A thirtieth aspect of the invention, depending on any of the aspects from the twenty-fifth aspect to the twenty-ninth aspect, relates to a micro-needle for the transdermal administration of active molecules and/or for sampling biological fluids, wherein said polymeric material comprises a photo-cross linking polymer, in particular polyethylene glycol (PEG).

A thirty-first aspect of the invention, depending on any of the aspects from the twenty-fifth aspect to the thirtieth aspect, relates to a micro-needle for the transdermal administration of active molecules and/or for sampling biological fluids, wherein said polymeric material is added with a photoinitiator, in particular Darocur or Irgacure or LAP.

A thirty-second aspect of the invention, depending on any of the aspects from the twenty-fifth aspect to the thirty-first aspect, relates to a micro-needle for the transdermal administration of active molecules and/or for sampling biological fluids, wherein said polymeric material is added with a photosensitive polymer or photosensitive compound.

A thirty-third aspect of the invention, depending on any of the aspects from the twenty-fifth aspect to the thirty-second aspect, relates to a micro-needle for the transdermal administration of active molecules and/or for sampling biological fluids, wherein said polymeric material is added with metal particles, preferably with particles of a noble metal, even more preferably with gold particles.

A thirty-fourth aspect of the invention, depending on any of the aspects from the twenty-fifth aspect to the thirty-third aspect, relates to a micro-needle for the transdermal administration of active molecules and/or for sampling biological fluids, wherein said polymeric material is added with an active ingredient.

A thirty-fifth aspect of the invention relates to a device for the transdermal administration of active molecules and/or for sampling of biological fluids, comprising at least one micro-needle according to any of the aspects from the twenty-fifth aspect to the thirty-fourth aspect, and a support element, said at least one micro-needle extending from one surface of said support element moving away from said support element, wherein in particular said surface of said support element has an opening at said micro-needle, optionally the shape of said at least one opening being substantially identical to the shape of the base of said at least one micro-needle and/or the diameter or diagonal of said at least one opening having an extension substantially identical to the extension of the diameter or of the diagonal of the base of said at least one micro-needle.

A thirty-sixth aspect of the invention, depending on the thirty-fifth aspect, relates to a device for the transdermal administration of active molecules and/or for sampling biological fluids, comprising a plurality of micro-needles, each micro-needle of said plurality being in accordance with one any of the aspects from the twenty-fifth aspect to the thirty-fourth aspect, said micro-needles extending from said surface of said support element moving away from said support element, said micro-needles being positioned on said surface of said support element depending on a predetermined regular and/or orderly arrangement.

A thirty-seventh aspect of the invention, depending on the thirty-fifth aspect or on the thirty-sixth aspect, relates to a device for the transdermal administration of active molecules and/or for sampling of biological fluids, wherein an active ingredient is contained in the cavity of said micro-needle and/or in the cavities of said micro-needles.

A thirty-eighth aspect of the invention, dependent on any of the aspects from the thirty-fifth aspect to the thirty-seventh aspect, relates to a device for the transdermal administration of active molecules and/or for sampling biological fluids, further comprising at least one micro-fluidic circuit and/or at least one micro-duct and/or at least one micro-reservoir in fluid communication with the cavity of said micro-needle and/or with the cavities of said micro-needle.

A thirty-ninth aspect of the invention, depending on any of the aspects from the thirty-fifth aspect to the thirty-eighth aspect, relates to a device for the transdermal administration of active molecules and/or for sampling biological fluids, wherein said surface of said support element is flexible.

A fortieth independent aspect of the present invention relates to a method for the optical activation of the release of an active ingredient by means of a micro-needle, said micro-needle being made of a polymeric material added with a photosensitive polymer or with a photosensitive compound, a blind cavity being defined in said micro-needle and containing said active ingredient, comprising the step of:
exposing said micro-needle to a coherent radiation having a predetermined wavelength such to energize said photosensitive polymer or said photosensitive compound, said coherent radiation being preferably in the near infrared field.

A forty-first independent aspect of the present invention relates to a method for the thermal activation of the release of an active ingredient by means of a micro-needle, said micro-needle being made of a polymeric material added with metal particles, preferably particles of a noble metal, even more preferably with gold particles, a blind cavity being defined in said micro-needle and containing said active ingredient, comprising the step of:
exposing said micro-needle to a coherent radiation having a predetermined wavelength such to heat by radiation said metallic particles, said coherent radiation being preferably in the near infrared field.

The inventive characteristics of the above listed aspects will become clearer in the following detailed description, wherein reference will be made to the following figures:

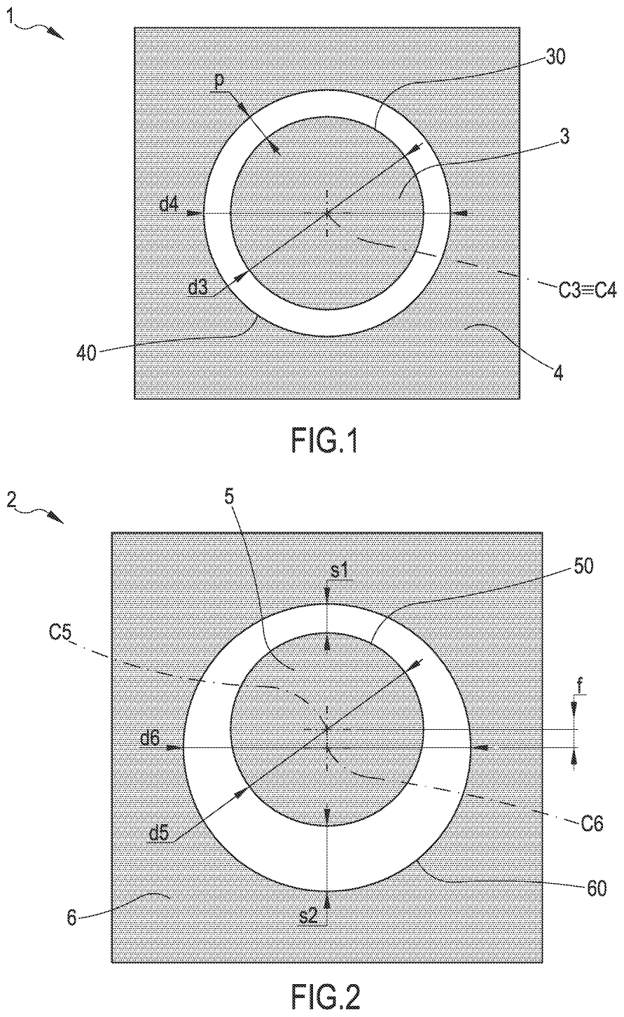

FIG. 1 and FIG. 2 represent two embodiments of photolithographic masks, usable in the method for obtaining at least one micro-needle for the transdermal administration of active molecules and/or for sampling biological fluids according to the present invention;

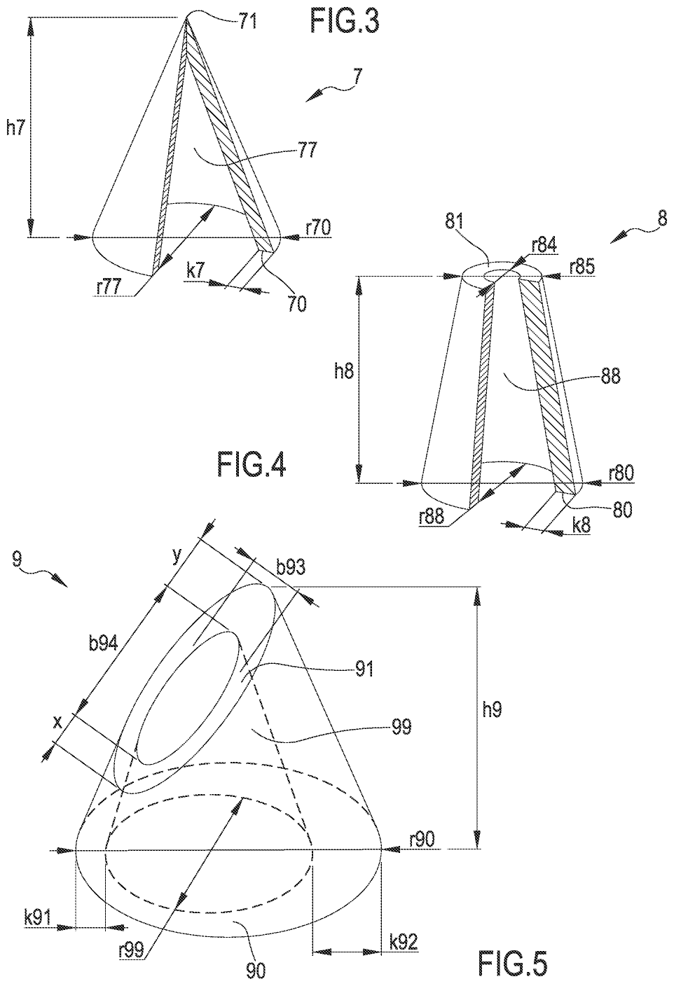

FIG. 3, FIG. 4 and FIG. 5 represent three embodiments of micro-needles for the transdermal administration of active molecules and/or for sampling biological fluids according to the present invention, in particular the micro-needles in FIG. 3 and in FIG. 4 being obtainable by means of the photolithographic mask in FIG. 1, the micro-needle in FIG. 5 being obtainable by means of the photolithographic mask in FIG. 2.

The present invention relates to a method for obtaining at least one micro-needle for the transdermal administration of active molecules and/or for sampling biological fluids, as well as the micro-needle obtained by this method. The micro-needle according to the present invention (which may have a symmetrical or asymmetrical shape) typically shows a cavity in its interior (which may be a blind cavity or a through cavity). The micro-needle can be advantageously integrated in a biomedical device, for topical use or for systemic use, which can advantageously integrate also micro-fluidic circuits for the adduction to the micro-needle of liquid substances or solutions of soluble substances or for the preservation of sampled quantities of biological fluids (blood, sweat, lymph, saliva, tears, interstitial fluid, and so on). The micro-needle according to the present invention is advantageously of an organic nature.

The embodiment according to the present invention is based on photolithography, by means of which a polymer or a polymeric mixture in the liquid phase hardens so as to assume a predetermined geometry. The manufacture of a micro-needle by photolithography is extremely advantageous, as it is very quick and cost-effective. Photolithography can easily be used on an industrial scale, with constant and repeatable results. Advantageously, the micro-needle according to the present invention is formed on the surface of a support element of a biomedical device.

Photolithography employs a photolithographic mask that is interposed between the photo-cross linking polymer to be hardened and a source of energy radiation capable of causing the hardening thereof. Advantageously, energy radiation is a UV (ultraviolet) radiation. As a photo-cross linking polymer, PEG (polyethylene glycol) can be used, having adequate transparency and appearing as a viscous liquid at room temperature. The photo-cross linking polymer can advantageously be added with a photocatalyst compound which plays the role of photoinitiator, in particular Darocur (2-Hydroxy-2-methyl-1-phenyl-propan-1-one) or Irgacure or LAP. The photo-cross linking polymer and photocatalyst compound form a photoresist hybrid polymeric mixture, wherein the photocatalyst compound has the function of triggering (by means of free radical polymerization) the cross-linking of the photo-cross linking polymer. The photocatalyst compound is present in the mixture according to a predetermined concentration: if Darocur is used, an adequate concentration of this compound in PEG is about 2% volume/volume. A hybrid photoresist polymer mixture of the photocatalyst compound (e.g. Darocur) in PEG behaves as a negative photoresist solution that branches when hardened if exposed to a UV source.

Photolithography allows to obtain the micro-needle by means of cross-linking and the consequent hardening of the photo-cross linking polymer and/or of the photoresist hybrid polymeric mixture, under irradiation conditions by means of an energetic radiation, in particular by means of a UV radiation. Once the desired geometry of the micro-needle is obtained, the irradiation is interrupted, thus ending the cross-linking process. The photo-cross linking polymer and/or the photoresist hybrid polymeric mixture remained in liquid phase (and which therefore did not undergo hardening) are finally removed by washing the micro-needle, in particular in deionized water. As an alternative to washing, the removal of the photoresist polymer and/or of the non-hardened hybrid photoresist mixture can take place by incineration in plasma.

According to the present invention, the photolithographic mask used for obtaining the micro-needle has a geometry such to obtain the cavity (blind or through) inside the micro-needle without requiring additional operations with respect to photolithography, such as an operation of removal of hardened polymeric material. The photolithographic mask according to the present invention shows a peculiar configuration, which allows to generate in the photo-cross linking polymer in liquid phase, irradiated through the photolithographic mask, a peripheral shadow area, a central shadow area and a lighting area confined between the peripheral shadow area and the central shadow area. The provision of a lighting zone confined between two shadow areas allows the cavity to be obtained inside the micro-needle during photolithography.

Since in photolithography the design of the appropriately illuminated photolithographic mask is transferred to the structure that is obtained by photolithography, it is advisable to use a material with a high dimensional stability for the photolithographic mask, for example a nickel/chromium alloy (characterized by reduced susceptibility to thermal deformations). It is also advisable to define the design of the photolithographic mask providing a very high resolution (and therefore very small tolerances, for example ±1 micrometer). The photolithographic mask, having to generate shaded areas in the irradiated photo-cross linking polymer, appears opaque so as to intercept the UV radiation. The photolithographic mask is advantageously associated with a plate which acts as a support structure for the photolithographic mask. The plate is transparent, hence allowing the UV radiation to pass through it in the regions not covered by the photolithographic mask. A proper constituent material for the plate is quartz, since it combines the desired permeability to UV radiation with significant stiffness and significant stability. Rigidity and stability in fact allow the plate (and consequently the associated mask) to be properly manipulated. Photolithography is a process suited to high automation, so that the plate can be aligned by means of an instrument known in the field with the name of “mask aligner”, which appropriately positions the plate (and consequently the mask) with regard to the UV source and to the photo-cross linking polymer in liquid phase, allowing the construction of the structure as much as possible according to the desired geometry.

The photolithographic masks represented in the plan views in FIG. 1 and FIG. 2 represent examples, shown for explanatory but not limitative purposes, of photolithographic masks suitable to be used for the implementation of the method according to the present invention. In the photolithographic masks shown in FIG. 1 and in FIG. 2, the areas of impermeability to UV radiation are distinguished from the areas of permeability to UV radiation (that is to say from those areas of the plate not covered by the photolithographic mask) as the latter is filled with a dot pattern, while the permeability areas are left blank.

In particular, the photolithographic mask 1 shown in FIG. 1 represents an example of a photolithographic mask suited for obtaining a hollow micro-needle with a substantially symmetrical shape, while the photolithographic mask 2 shown in FIG. 2 represents an example of photolithographic mask suitable for use in the manufacture of an asymmetrical hollow micro-needle.

The photolithographic mask 1 in FIG. 1 comprises a peripheral region of impermeability 4 to energy radiation, in particular to UV radiation, and a central region of impermeability 3 to energy radiation, in particular to UV radiation, the peripheral region 4 being distinct and separate from the central region 3. The peripheral region of impermeability 4 is suitable to generate the peripheral shadow area, while the central region of impermeability 3 is suitable to generate the central shadow area. The photolithographic mask 1 therefore follows a design wherein two profiles are defined: an outer profile 40 which internally delimits the peripheral region 4 and an inner profile 30 which externally delimits the central region 3. The outer profile 40 is therefore capable of separating a region of impermeability to its own exterior from a region of permeability within itself. On the contrary, the inner profile 30 is capable of separating a region of impermeability within itself from a region of permeability to its own exterior. The outer profile 40 entirely encloses the inner profile 30.

In the embodiment of the photolithographic mask 1 shown in FIG. 1 the outer profile 40 and the inner profile 30 are both circular profiles.

Consequently, the permeability region of the photolithographic mask 1 has the shape of a circular crown. However, the circular shape of the outer profile 40 and of the inner profile 30 must be interpreted as a purely exemplary characteristic of the photolithographic mask 1, as according to the present invention, outer profiles and inner profiles of different shapes are also possible, for example elliptical profiles or polygonal profiles (for example an octagonal profile). Advantageously, the outer profile has a shape coinciding with the shape of the inner profile, however the present invention is not to be considered limited in this sense, since it is for example possible to provide an octagonal shape for the outer profile and a circular shape for the inner profile.

A peculiar characteristic of the photolithographic mask 1, whereby it can be suitably used for obtaining a substantially symmetrical shaped hollow micro-needle, is the substantial concentricity between the outer profile 40 and the inner profile 30. This substantial concentricity allows the region of permeability of the photolithographic mask 1 to have a substantially constant extension throughout its development.

In FIG. 1 the references d4 and d3 indicate the characteristic dimensions of the outer profile 40 and of the inner profile 30. The characteristic dimension d4 of the outer profile 40 plays a role in defining the width of the base of the micro-needle obtained by means of the photolithographic mask 1. The characteristic dimension d3 of the inner profile 30 plays instead a role in defining the width of the cavity obtained inside the micro-needle. The difference between the characteristic dimension d4 of the outer profile 40 and the characteristic dimension d3 of the inner profile 30 finally plays a role in defining the thickness of the walls of the micro-needle.

In the embodiment of the photolithographic mask 1 shown in FIG. 1, wherein the region of permeability of the photolithographic mask 1 has the shape of a circular crown, the distance p between the outer profile 40 and the inner profile 30 defines the extension of the region of permeability and can be calculated using the following formula:

p=(d4−d3)/2

Since in the embodiment of the photolithographic mask 1 represented in FIG. 1 the outer profile 40 is circular and the inner profile 30 is also circular, it is that the characteristic dimension d4 coincides with the diameter of the outer profile 40, while the characteristic dimension d3 coincides with the diameter of the inner profile 30. In the case of polygonal profiles, the respective diagonals can be suitably considered as characteristic dimensions for the outer profile and for the inner profile. By way of example, a possible sizing of the photolithographic mask 1 according to the embodiment of the photolithographic mask 1 shown in FIG. 1 is reported below:
characteristic dimension d4 (diameter) of the outer profile 40 comprised between 100 micrometers and 910 micrometers, preferably between 300 micrometers and 900 micrometers, even more preferably about 500 micrometers;
characteristic dimension d3 (diameter) of the inner profile 30 comprised between 90 micrometers and 900 micrometers, preferably between 100 micrometers and 700 micrometers, even more preferably about 300 micrometers;
distance p between the outer profile 40 and the inner profile 30 of between 10 micrometers and 200 micrometers, preferably between 60 micrometers and 140 micrometers, even more preferably about 100 micrometers.

By exposing the photo-cross linking polymer to UV radiation with the interposition of the photolithographic mask 1, the UV radiation passing through the region of permeability (ie the circular crown between the outer profile 40 and the inner profile 30 in the embodiment shown in FIG. 1) is first refracted by the constituent material of the plate (for example quartz) and then by the layers of photo-cross linking polymer below the photolithographic mask 1 which have been hardened and therefore solidified, while maintaining their transparency to UV radiation. As a result of the refraction, the UV radiation, after passing through the region of permeability, is diverted inwards.

Consequently, the central shadow area penetrates into the photo-cross linking polymer in liquid phase (contained in a container, preferably in silicone, positioned below the photolithographic mask 1) for a limited extension, defined by the geometric characteristics of the photolithographic mask 1 and from the optical properties of the photo-cross linking polymer and/or the hybrid photoresist polymer mixture. In particular, since the outer profile 40 and the inner profile 30 are both circular, it results that the central shadow area is right cone-shaped and the maximum penetration of the central shadow area in the photo-cross linking polymer in liquid phase has an extension which is equal to the height of the right cone.

If the outer profile and the inner profile had both polygonal shape, the central shadow area would be regular pyramid-shaped and the penetration of the central shadow area in the photo-reticulating liquid polymer would have an extension equal to the height of the regular pyramid.

Therefore it is possible to obtain, by means of a photolithographic mask characterized by a substantial concentricity between the outer profile 40 and the inner profile 30 (such as for example the photolithographic mask 1 shown in FIG. 1), a straight cone (or a regular pyramid) shaped hollow micro-needle or a right truncated cone (or a truncated regular pyramid) shaped hollow micro-needle, depending on the energy supplied to the photo-cross linking polymer by means of energy radiation, in particular UV radiation. Since the energy of a radiation is given by the product between the power of the radiation and the time of exposure to the radiation, it results that:
in the case UV radiation is used with a predetermined and constant power, a straight cone (or a regular pyramid) shaped micro-needle, comprising a conical (or pyramidal) and substantially coaxial internal blind cavity, can be obtained by keeping the photo-cross linking polymer exposed to UV radiation up to a time corresponding to the time required by the UV radiation of the predetermined power to harden the photo-cross linking polymer to a depth equal to the penetration of the central shadow area in the photo-cross linking polymer;
in the case UV radiation is used with a predetermined and constant power, a truncated right cone (or a truncated regular pyramid) shaped micro-needle, comprising a substantially coaxial internal cavity in the shape of a truncated cone (or truncated pyramid), can be obtained by interrupting the exposure of the photo-cross linking polymer before reaching the time required by the UV radiation of the predetermined power to harden the photo-cross linking polymer to a depth equal to the penetration of the central shadow area in the photo-cross polymer;
if the UV source is such to allow a modulation of the power of the UV radiation, intending to keep the exposure time constant, with a first power of the UV radiation it is possible to obtain a conical (or pyramidal) shaped micro-needle comprising a conical (or pyramidal) and substantially coaxial internal blind cavity, while at a second power of the UV radiation (suitably lower than the first power) it is possible to obtain a truncated cone-shaped (or truncated pyramid-shaped) micro-needle, comprising a substantially coaxial internal cavity in the shape of a truncated cone (or truncated pyramid).

A first micro-needle 7 for the transdermal administration of active molecules and/or for sampling biological fluids which can be obtained by means of the photolithographic mask 1 shown in FIG. 1 (in particular by extending the exposure of the photo-cross linking polymer to the UV radiation to a predetermined duration and/or setting the power of the UV source above a predetermined power) is represented in the partially sectioned axonometric view in FIG. 3. The micro-needle 7 is right cone-shaped. Inside the micro-needle 7 a cavity 77 is defined, which is substantially coaxial and also right cone-shaped.

The cavity 77 of the micro-needle 7 is a blind cavity. The micro-needle 7 develops starting from the base 70 (substantially in the shape of a circular crown) retaining a substantially constant thickness, until it reaches the vertex 71. FIG. 3 shows the main geometric parameters characterizing the micro-needle 7 (right cone-shaped) with the blind cavity 77:
the reference h7 identifies the height of the micro-needle 7, which can be between 200 micrometers and 2000 micrometers, preferably between 900 micrometers and 1300 micrometers, even more preferably about 1100 micrometers;
the reference r70 identifies the characteristic size (in particular the diameter) of the base 70 of the micro-needle 7, which can be between 100 micrometers and 900 micrometers, preferably between 300 micrometers and 700 micrometers, even more preferably about 500 micrometers;
the reference r77 identifies the characteristic dimension (in particular the diameter) of the cavity 77 of the micro-needle 7 at the base 70, which can be between 80 micrometers and 880 micrometers, preferably between 180 micrometers and 580 micrometers, even more preferably about 300 micrometers;
the reference k7 identifies the thickness of the walls of the micro-needle 7, which can be between 10 micrometers and 200 micrometers, preferably between 60 micrometers and 140 micrometers, even more preferably about 100 micrometers.

The micro-needle 7, having a blind cavity 77, is particularly suited for the transdermal administration of active molecules. The blind cavity 77 is in fact able to act as a micro-reservoir that can be filled with an active ingredient (typically in liquid phase or in solution).

Following the application to a patient of the micro-needle 7 or of a device for the transdermal administration of active molecules integrating the micro-needle 7, the active ingredient is released progressively, in a time depending on the permeability of the walls of the micro-needle 7 to the molecules of the active ingredient and/or from the hydrophobic or hydrophilic nature of the molecules of the active ingredient.

A second micro-needle 8 for the transdermal administration of active molecules and/or for sampling biological fluids which can be obtained by means of the photolithographic mask 1 shown in FIG. 1 (in particular by interrupting the exposure of the photo-cross linking polymer to the UV radiation before a predetermined duration and/or setting the power of the UV source below a predetermined power) is shown in the partially sectioned axonometric view in FIG. 4. The micro-needle 8 is truncated cone-shaped. Inside the micro-needle 8 a cavity 88 is defined which is substantially coaxial and also truncated cone-shaped. The cavity 88 of the micro-needle 8 is a through cavity. The micro-needle 8 develops between two bases (larger base 80 and smaller base 81), both substantially in the shape of a circular crown. The thickness of the micro-needle 8 remains substantially constant throughout its development between the larger base 80 and the smaller base 81. FIG. 4 shows the main geometrical parameters distinguishing micro-needle 8 (straight truncated cone-shaped) with the through cavity 88:
the reference h8 identifies the height of the micro-needle 8, which can be between 200 micrometers and 2000 micrometers, preferably between 900 micrometers and 1300 micrometers, even more preferably about 1100 micrometers;
the reference r80 identifies the characteristic dimension (in particular the diameter) of the larger base 80 of the micro-needle 8, which can be between 100 micrometers and 900 micrometers, preferably between 300 micrometers and 700 micrometers, even more preferably about 500 micrometers;
the reference r85 identifies the characteristic size (in particular the diameter) of the smaller base 81 of the micro-needle 8, which can be comprised between 30 micrometers and 500 micrometers, preferably between 200 micrometers and 400 micrometers, even more preferably about 300 micrometers;
the reference r88 identifies the characteristic dimension (in particular the diameter) of the cavity 88 of the micro-needle 8 at the larger base 80, which can be between 80 micrometers and 880 micrometers, preferably between 180 micrometers and 580 micrometers, even more preferably about 300 micrometers;
the reference r84 identifies the characteristic dimension (in particular the diameter) of the cavity 88 of the micro-needle 8 at the lower base 81, which can be comprised between 10 micrometers and 480 micrometers, preferably between 80 micrometers and 280 micrometers, even more preferably about 180 micrometers;
the reference k8 identifies the thickness of the walls of the micro-needle 8, which can be between 10 micrometers and 200 micrometers, preferably between 60 micrometers and 140 micrometers, even more preferably about 100 micrometers.

The micro-needle 8, having a through cavity 88, is particularly suited for sampling biological fluids (blood, sweat, lymph, saliva, tears, interstitial fluid, and so on). In fact the through cavity 88 can act as a micro-conduct which can be traversed by biological fluids in a relatively rapid time. Following the application to a patient of the micro-needle 8 or of a device for sampling biological fluids integrating the micro-needle 8, the biological fluid (for example blood) taken from the patient reaches the site (for example a reservoir or a micro-reservoir) where it is sampled by easily passing through the cavity 88.

The photolithographic mask 2 in FIG. 2 comprises a peripheral region 6 of impermeability to energy radiation, in particular to UV radiation, and a central region 5 of impermeability to energy radiation, in particular to UV radiation, the peripheral region 6 being distinct and separated from the central region 5. The peripheral region 6 of impermeability is suitable to generate the peripheral shadow area, while the central impermeability region 5 is suitable to generate the central shadow zone. The photolithographic mask 1 therefore complies with a drawing wherein two profiles are defined: an outer profile 60 which internally delimits the peripheral region 6 and an inner profile 50 which delimits the central region 5 externally. The outer profile 60 is therefore capable of separating a region of impermeability to its exterior from a region of permeability within itself. On the contrary, the inner profile 50 is capable of separating a region of impermeability within itself from a region of permeability to its own exterior. The outer profile 60 entirely encloses the inner profile 50.

In the embodiment of the photolithographic mask 2 shown in FIG. 2, the outer profile 60 and the inner profile 50 are both circular profiles. However, the circular shape of the external profile 60 and of the internal profile 50 must be interpreted as a purely exemplary characteristic of the photolithographic mask 2, as according to the present invention, outer profiles and inner profiles of different shapes are also possible, for example elliptical profiles or polygonal profiles (for example an octagonal profile). Advantageously, the outer profile has a shape coinciding with the shape of the inner profile, however the present invention is not to be considered limited in this sense, since it is for example possible to provide an octagonal shape for the outer profile and a circular shape for the inner profile.

A peculiar characteristic of the photolithographic mask 2, such that it can be suitably used for the manufacture of an asymmetric hollow micro-needle, is the spacing between the center of curvature C6 of the outer profile 60 and the center of curvature C5 of the inner profile 50, so that the outer profile 60 and the inner profile 50 are not concentric with each other and the region of permeability of the photolithographic mask 2 does not have a constant extension along its own development. The region of permeability of the photolithographic mask 2 has a symmetrical shape, the axis of symmetry of the permeability region coinciding with the straight line passing through the center of curvature C6 of the outer profile 60 and through the center of curvature C5 of the inner profile 50.

In FIG. 2 the references d6 and d5 indicate the characteristic dimensions of the outer profile 60 and of the inner profile 50 respectively. The extension of the region of permeability of the photolithographic mask 2 depends on these characteristic dimensions, as well as on the distance f between the center of curvature C6 of the outer profile 60 and the center of curvature C5 of the inner profile 50. In particular, the extension of the region of permeability of the photolithographic mask 2 varies gradually and progressively between a minimum distance s1 and a maximum distance s2, related to each other by the following formulas:

$$\{ \begin{matrix}s 2 + s 1 = d 6 - d 5 \\ f = ( s 2 - s 1 ) / 2\end{matrix}$$

Since in the embodiment of the photolithographic mask 2 shown in FIG. 2 the outer profile 60 is circular and the inner profile 50 is also circular, it is that the characteristic dimension d6 coincides with the diameter of the outer profile 60, while the characteristic dimension d5 coincides with the diameter of the inner profile 50. In the case of polygonal profiles, the respective diagonals can be suitably considered as the characteristic dimensions for the outer profile and for the inner profile. By way of example, a possible sizing of the photolithographic mask 2 according to the embodiment of the photolithographic mask 2 shown in FIG. 2 is reported below:
characteristic dimension d6 (diameter) of the outer profile 60 comprised between 100 micrometers and 910 micrometers, preferably between 300 micrometers and 900 micrometers, even more preferably about 500 micrometers;
characteristic dimension d5 (diameter) of the inner profile 50 comprised between 90 micrometers and 900 micrometers, preferably between 100 micrometers and 700 micrometers, even more preferably about 300 micrometers;
distance f between the geometric center C6 of the outer profile 60 and the geometric center C5 of the inner profile 50 comprised between 10 micrometers and 200 micrometers, preferably between 30 micrometers and 50 micrometers, even more preferably about 40 micrometers;
minimum distance s1 between the outer profile 60 and the inner profile 50 comprised between 10 micrometers and 180 micrometers, preferably between 40 micrometers and 120 micrometers, even more preferably about 60 micrometers;
maximum distance s2 between the outer profile 60 and the inner profile 50 of between 30 micrometers and 200 micrometers, preferably between 80 micrometers and 160 micrometers, even more preferably about 140 micrometers.

Due to the eccentricity of the central region 5 with respect to the peripheral region 6, the central shadow area that is created by exposing the photo-cross linking polymer to the UV radiation with the interposition of the photolithographic mask 2, due to the refraction phenomenon is oblique cone-shaped (in case the inner profile 50 is circular-shaped) or irregular pyramid-shaped (in case the internal profile 50 is polygonal-shaped), the axis of this oblique cone or of this irregular pyramid presenting a predetermined inclination with respect to the axis of the peripheral shadow area. The inclination of the axis of the central shadow area with respect to the axis of the peripheral shadow area is determined by the distance f between the center of curvature C6 of the outer profile 60 and the center of curvature C5 of the inner profile 50 in the photolithographic mask 2.

Thus it is possible to obtain, by means of a photolithographic mask characterized by the eccentricity of the inner profile 50 with respect to the outer profile 60 (such as for example the photolithographic mask 2 shown in FIG. 2), an oblique truncated cone-shaped hollow micro-needle (or an irregular truncated pyramid-shaped hollow micro-needle).

A micro-needle 9 for the transdermal administration of active molecules and/or for sampling of biological fluids obtained by means of the photolithographic mask 2 shown in FIG. 2 (in particular by interrupting the exposure of the photo-cross linking polymer to the UV radiation before a predetermined duration and/or setting the power of the UV source below a predetermined power) is shown in the axonometric view of FIG. 5, wherein the non-visible contours are delineated by dashed lines. The micro-needle 9 is oblique truncated cone-shaped. Inside the micro-needle 9 a cavity 99 is defined, which is also oblique truncated cone-shaped. The cavity 99 of the micro-needle 9 is a through cavity.

The inclination of the walls of the micro-needle 9 depends on the inclination of the axis of the central shadow area with respect to the axis of the peripheral shadow area that are created during the photolithography operation by which the micro-needle 9 is obtained. Therefore the inclination of the walls of the micro-needle 9 is determined by the distance f between the center of curvature C6 of the outer profile 60 and the center of curvature C5 of the inner profile 50 in the photolithographic mask 2. Accordingly, the present invention makes it possible to obtain the desired inclination for the micro-needle 9, suitably adapting the geometry of the photolithographic mask 2 used for the photolithography of the micro-needle 9, in particular by suitably setting the distance f between the center of curvature C6 of the outer profile 60 and the center of curvature C5 of the inner profile 50.

Due to the eccentricity of the inner profile 50 with respect to the outer profile 60 in the photolithographic mask 2, the development of the walls of the micro-needle 9 depends on the extension of the region of permeability of the photolithographic mask 2 from which these walls originate, hence the height of the micro-needle 9 has a minimum value at the minimum distance s1 and a maximum value at the maximum distance s2.

The micro-needle 9 develops between two bases not parallel to each other, the smaller base 91 lying on a plane incident to the projective plane of the larger base 90. From a strictly geometric point of view, it can therefore be assumed that the smaller base 91 of the micro-needle 9 is obtained by cutting a cone whose base coincides with the larger base 90 of the micro-needle 9 along a plane not orthogonal to the axis of the cone. As known, by cutting a cone along a plane that is not orthogonal to the axis, the obtained flat section has the shape of an ellipse. Therefore in the micro-needle 9, while the larger base 90 is substantially circumferential, the smaller base 91 is substantially an ellipse. Similarly, also the through cavity 99 of the micro-needle 9 develops between the larger base 90 and the smaller base 91 taking the shape of a circular opening at the larger base 90 and the shape of an elliptical opening at the smaller base 91. In particular, the elliptical opening at the smaller base 91 is oriented in such a way that the major axis b94 (on which the two foci lie) coincides with the projection (on the projective plane of the smaller base 91) of the straight line passing both through the center of curvature of the outer profile 60 and through the center of curvature of the inner profile 50, while the minor axis b93 coincides with the projection (on the projective plane of the smaller base 91) of the straight line passing through the center of curvature of the inner profile 50 and orthogonal to the straight line both for the center of curvature of the outer profile 60 and for the center of curvature of the inner profile 50.

The thickness of the walls of the micro-needle 9 varies according to the orientation of the walls with respect to the straight line passing both through the center of curvature of the outer profile 60 and through the center of curvature of the inner profile 50 (the more eccentric the center of curvature of the inner profile 50 with respect to the center of curvature of the outer profile 60, the more noticeable the variation will be), while it remains substantially constant in case of variations of the height of the micro-needle 9 only. In particular, the thickness of the walls of the micro-needle 9 has a minimum value at the minimum distance s1 and a maximum value at the maximum distance s2.

Further variable according to the orientation of the walls with respect to the straight line passing both through the center of curvature of the outer profile 60 and through the center of curvature of the inner profile 50 (this variation being more accentuated the more the center of curvature of the inner profile 50 is eccentric with respect to the center of curvature of the outer profile 60) is the inclination of the walls of the micro-needle 9 with respect to the projective plane of the larger base 90 and the inclination of the cavity 99 with respect to the projective plane of the larger base 90. In particular, these inclinations have a respective maximum value at the minimum distance s1 and a respective minimum value at the maximum distance s2.

In view of the above, with the references k92 and k91 respectively the maximum and minimum thicknesses of the walls of the micro-needle 9 and with the y and x references respectively the maximum and minimum distances (measured on the projective plane of the smaller base 91) between the walls of the micro-needle 9 and the elliptical opening, the following relation applies:

x/y=k91/k92

FIG. 5 shows the main geometric parameters that distinguish the micro-needle 9 (truncated oblique cone-shaped) with the through cavity 99:
the reference h9 identifies the maximum height of the micro-needle 9, which can be between 600 micrometers and 2400 micrometers, preferably between 1100 micrometers and 1500 micrometers, even more preferably about 1200 micrometers;
the reference r90 identifies the characteristic dimension (in particular the diameter) of the larger base 90 of the micro-needle 9, which can be between 100 micrometers and 900 micrometers, preferably between 300 micrometers and 700 micrometers, even more preferably about 500 micrometers;
the reference r99 identifies the characteristic dimension (in particular the diameter) of the cavity 99 of the micro-needle 9 at the larger base 90, which can be between 80 micrometers and 880 micrometers, preferably between 180 micrometers and 580 micrometers, even more preferably about 300 micrometers;
the reference k92 identifies the maximum thickness of the walls of the micro-needle 9, which can be between 30 micrometers and 240 micrometers, preferably between 40 micrometers and 180 micrometers, even more preferably about 120 micrometers;
the reference k91 identifies the minimum thickness of the walls of the micro-needle 9, which can be between 10 micrometers and 180 micrometers, preferably between 20 micrometers and 120 micrometers, even more preferably about 80 micrometers.

The micro-needle 9, having a through cavity 99, is particularly designed to be used for sampling biological fluids (blood, sweat, lymph, saliva, tears, interstitial fluid, and so on). The through cavity 99 is in fact suitable to function as a micro-duct which can be traversed by biological fluids in a relatively rapid time. Following the application to a patient of the micro-needle 9 or of a device for sampling biological fluids integrating the micro-needle 9, the biological fluid (for example blood) taken from the patient reaches the site (for example a reservoir or a micro-reservoir) where it is sampled by easily passing through the cavity 99.

With respect to the micro-needle 9, it is to be emphasized that the inclination of the smaller base 91 with respect to the projective plane of the larger base 90 is extremely advantageous, since it allows to arrange, at the end of the micro-needle 9, a cutting tip which easily penetrates in the stratum corneum of the patient's skin, thus further minimizing the possible sensation of pain deriving from the indentation of the micro-needle 9.

In an advantageous embodiment of the present invention, the molecular weight of the photo-cross linking polymer from which the manufacture by photolithography of the micro-needle takes place can be modulated, so as to obtain more or less large nano-cavities in the micro-needle. In particular, this modulation occurs using PEG at high or low molecular weight. The molecular weight modulation of the photo-cross linking polymer is most useful when the micro-needle is provided with a blind cavity and it is used for the release of an active ingredient, in order to suitably regulate the release rate of the active ingredient molecules through the micro-needle. In fact, if large nano-cavities are obtained in the micro-needle, the micro-needle has morphological characteristics such as to obtain a relatively high release rate of the molecules of the active ingredient. If, on the other hand, small nano-cavities are obtained in the micro-needle, the micro-needle has morphological characteristics such as to obtain a relatively low release speed of the molecules of the active ingredient.

In an advantageous embodiment of the present invention, the wettability of the photo-cross linking polymer from which the fabrication by photolithography of the micro-needle takes place can be modulated so as to confer the desired surface chemistry characteristics to the micro-needle. In particular, the micro-needle can have hydrophobic nature or hydrophilic nature. The modulation of the molecular weight of the photo-cross linking polymer is most useful when the micro-needle is provided with a blind cavity and it is used for the release of an active ingredient, in order to suitably regulate the release speed of the active ingredient molecules through the micro-needle, based on the polarization of the molecular structure of the active ingredient. The choice of the hydrophobic or hydrophilic behaviour of the micro-needle depends on whether one intends to obtain agreement between the active ingredient and the micro-needle (both of hydrophobic nature or both of hydrophilic nature) or discordance between the active ingredient and the micro-needle (active ingredient of hydrophobic nature and micro-needle of hydrophilic nature, or active ingredient of hydrophilic nature and micro-needle of hydrophobic nature) according to the desired release speed of the active ingredient through the micro-needle.

In an advantageous embodiment of the present invention, the photo-cross linking polymer (for example PEG) starting from which the manufacture by photolithography of the micro-needle takes place is added with an active ingredient. Once the micro-needle has been applied to a patient, the active ingredient is released from the micro-needle to the patient.

In an advantageous embodiment of the present invention, the micro-needle is configured to allow the release of an active ingredient by optical activation. According to this embodiment, the cavity of the micro-needle is a blind cavity filled with the active ingredient and the photo-cross linking polymer (for example PEG), starting from which the fabrication by photolithography of the micro-needle takes place, is added with a photosensitive polymer or with a photosensitive compound (for example a pigment). The micro-needle is configured to be usually impermeable to the molecules of the active ingredient. However, when the photosensitive polymer or the photosensitive compound are energized, in particular by exposing the micro-needle to a predetermined radiation (preferably in the near infrared field) capable of causing the resonance of the molecules of the photosensitive polymer or of the photosensitive compound, the behavior of the micro-needle changes, becoming permeable to the molecules of the active ingredient. This can occur, for example, because the heat transmitted by the photosensitive polymer or by the photosensitive compound after being energized is able to increase the fluidity of the active ingredient loaded in the cavity in gel form, so that it can move by capillarity through the nano-cavities of the micro-needle and thus it can be released from the micro-needle.

In an advantageous embodiment of the present invention, the micro-needle is configured to allow the release of an active ingredient by thermal activation. According to this embodiment, the cavity of the micro-needle is a blind cavity filled with the active ingredient and the photo-cross linking polymer (for example PEG), from which the fabrication by photolithography of the micro-needle takes place, is added with metal particles, preferably with particles of a noble metal, even more preferably with gold particles. The micro-needle is configured to be usually impermeable to the molecules of the active ingredient. However, when the metal particles are heated, in particular by exposing the micro-needle to a predetermined radiation (preferably in the near infrared field) capable of increasing the temperature of the metal particles by irradiation, the behavior of the micro-needle changes, becoming permeable to the molecules of the active ingredient. This can happen for example because the heat that is transmitted by the metallic particles after their heating by irradiation is able to make more fluid the active ingredient loaded in the cavity in gel form, so that it can to move by capillarity through the nano-cavities of the micro-needle and thus it can be released from the micro-needle.

The present invention further relates to a method for selectively releasing an active ingredient by means of a micro-needle (preferably a micro-needle of polymeric material, for example obtained in PEG by photolithography) or by means of a device for the transdermal administration of active molecules comprising a micro-needle or a plurality of micro-needles. This method requires that the active ingredient to be released is contained within a blind cavity of the micro-needle. This method also requires the presence of dispersed molecules of a photosensitive polymer or molecules of a photosensitive compound (for example molecules of a pigment) and/or the presence of dispersed metal particles, preferably particles of a noble metal, even more preferably gold particles, in the structure of the micro-needle.

The method according to the present invention for selectively releasing an active ingredient requires that the release of the molecules of the active ingredient through the micro-needle is conditioned by the exposure of the micro-needle to a dedicated radiation (the micro-needle remains in fact substantially impermeable to the molecules of the active ingredient unless exposure to a dedicated radiation occurs). In particular, the method according to the present invention for selectively releasing an active ingredient comprises the specific phase of exposing the micro-needle to a dedicated radiation, particularly to a coherent radiation of a predetermined wavelength. Advantageously, the radiation for making the micro-needle permeable to the molecules of the active ingredient is chosen in the near infrared field.

In the case that molecules of a photosensitive polymer or molecules of a photosensitive compound are dispersed in the structure of the micro-needle, the exposure of the micro-needle to the dedicated radiation causes the resonance of the molecules of the photosensitive polymer or of the photosensitive compound, whereas, in the case that metal particles are dispersed in the structure of the micro-needle, exposure of the micro-needle to the dedicated radiation causes the heating by irradiation of the metal particles. Either through the resonance of the molecules of the photosensitive polymer or the photosensitive compound, or by irradiation heating of the metal particles, the energy necessary for the activation of the release of the active ingredient is therefore conferred to the micro-needle.

The present invention further relates to a device for the transdermal administration of active molecules and/or for sampling biological fluids. The device comprises a support element and one or more micro-needles made by photolithography on a surface of this support element, so as to extend away from this surface of the support element, the micro-needles being particularly made of polymeric material. Given that the support element is placed in contact with the patient's skin (to allow the insertion of the micro-needles) when the device according to the present invention is in use and given that the human body has a strongly irregular geometry, the device is advantageously flexible (or at least the support element of the device is flexible), so as to adapt to the shape of the region of the human body where the device is applied. Adequate flexibility can be obtained, in addition to a suitable choice and/or a suitable additivation of the polymeric material constituting the support element, also by attributing to the support element a relatively reduced thickness (advantageously a thickness comprised between 300 micrometers and 2 millimeters, for example of about 1 millimeter).

In at least one of the micro-needles a cavity is defined which can be a blind cavity (in which case the device according to the present invention is particularly predisposed to be used for the transdermal administration of active molecules) or a through cavity (in which case the device according to the present invention is particularly designed to be used for sampling biological fluids). Advantageously, a (blind or through) cavity is defined in each of the device's micro-needles. Advantageously, the micro-needles of the device are made according to the same geometry; however it is not excluded that the micro-needles can be made according to different geometries and some can further feature a blind cavity and others a through cavity. Advantageously, the micro-needles are arranged on the support element depending on a predetermined regular and/or orderly arrangement (for example they can be aligned or staggered to each other, so as to form a plurality of substantially parallel rows).

The micro-needles described above (for example, the right cone-shaped micro-needle 7 shown in FIG. 3 defining a blind cavity 77 or the truncated right cone-shaped micro-needle 8 shown in FIG. 4 defining a through cavity 88 or the truncated oblique cone-shaped micro-needle 9 shown in FIG. 5 defining a through cavity 99) are all suitable to form part of the device for the transdermal administration of active molecules and/or for sampling biological fluids according to the present invention. Furthermore, each of the hollow micro-needles forming part of the device for the transdermal administration of active molecules and/or for sampling biological fluids according to the present invention can be given any of the characteristics previously described with reference to the micro-needles: for example the modulation of the nano-cavity size; the hydrophobic or hydrophilic nature; the suitability of cavities (if they are blind cavities) to serve as micro-reservoirs in which the active ingredient to be released can be stored; the additivation of the constituent polymeric material with an active ingredient and/or with a photosensitive polymer or a photosensitive compound and/or with metal particles, and so on.

Advantageously, the support element of the device according to the present invention for the transdermal administration of active molecules and/or for sampling biological fluids has an opening where each micro-needle is to be arranged. In particular, the characteristic size of the openings (e.g. the diameter, if the openings are cylindrical openings with a circular cross-section) is substantially equal to the characteristic size of the micro-needles which will then be made by photolithography where such openings are located. Therefore, wishing to obtain on the support element, micro-needles whose geometry reproduces the geometry of the micro-needle in FIG. 3, the characteristic dimension of the openings of the support element will substantially be coincident with the characteristic dimension (in particular the diameter) of the base 70 of the micro-needle 7. Moreover, wishing to obtain, on the support element, micro-needles whose geometry reproduces the geometry of the micro-needle of FIG. 4, the characteristic dimension of the openings of the support element will substantially be coincident with the characteristic dimension (in particular the diameter) of the larger base 80 of the micro-needle 8. Finally, wishing to obtain, on the support element, micro-needles whose geometry reproduces the geometry of the micro-needle of FIG. 5, the characteristic dimension of the openings of the support element it will substantially coincide with the characteristic dimension (in particular the diameter) of the larger base 90 of the micro-needle 9.

The support element of the device according to the present invention for the transdermal administration of active molecules and/or for sampling biological fluids is made of transparent material. In particular, the support element is made of PEG (that is to say in the same material which will then be used for the manufacture of micro-needles) through photolithography. It is possible to manufacture the support element first and subsequently the hollow micro-needles on this support element, by means of two distinct photolithographic operations. It is also possible, using a photolithographic mask of appropriate geometry, to obtain the support element and the hollow micro-needles during the same photolithographic operation.

A possible manufacturing method of the hollow micro-needles on the support element provides that a container, preferably in silicone, is filled up to its edges with the photo-cross linking polymer (for example PEG) in liquid phase and that the support element lies on the container so as to be in direct contact with the photo-cross linking polymer.

A photolithographic mask is then applied, whose characteristic size (diameter or diagonal) is advantageously between 20 mm and 360 mm, depending on the extension of the support element. The drawing at the base of the photolithographic mask takes into account the number and/or the distribution and/or the dimensions of the hollow micro-needles to be manufactured on the support element. Advantageously, the photolithographic mask is obtained by appropriately composing a plurality of individual elements (for example a plurality of individual elements each reproducing the drawing of the photolithographic mask 1 in FIG. 1 or the drawing of the photolithographic mask 2 in FIG. 2), so as to include a plurality of regions of permeability to the energy radiation (i.e. to the UV radiation), in particular a number of permeability regions corresponding to the number of hollow micro-needles to be manufactured, the arrangement of the permeability regions in the photolithographic mask (i.e. their alignment according to a plurality of rows) depending on the desired arrangement of the hollow micro-needles on the support element.

The photolithographic mask is suitably positioned with respect to the support element, advantageously by means of the “mask aligner” tool. In particular, the arrangement of the photolithographic mask is such that the regions of permeability defined in the photolithographic mask are substantially coaxial with the openings formed in the support element.

It is then exposed to energy radiation (for example to UV radiation), the exposure time being established according to the desired height of the micro-needles and/or of the desired configuration of the cavities (blind or through). In particular, the manufacture of micro-needles with blind cavities requires a longer exposure time than the manufacture of micro-needles with through cavities.

The device according to the present invention for the transdermal administration of active molecules and/or for sampling of biological fluids can integrate further elements in fluid communication with the cavities of the micro-needles, for example at least a microfluidic circuit and/or at least a micro-duct and/or at least one micro-reservoir. Advantageously, these further elements are advantageously realized on the support element by photolithography. Additionally, the device according to the present invention can integrate micro-actuators and/or micro-sensors (possibly with the corresponding control units), suitably assembled to the support element.

The device according to the present invention can be arranged for the transdermal administration of active molecules, for cosmetic or biomedical use, with the release of the active molecules being topical or systemic. By way of a purely explanatory and non-limiting example, a configuration can be considered wherein the device is provided with a plurality of micro-needles with blind cavities and with a micro-reservoir where the active ingredient is loaded (micro-needles and micro-reservoir being obtained by photolithography on opposite sides of the support element), the cavities in the micro-needles being in fluid connection with the micro-reservoir through micro-ducts. In this configuration, the release of the active ingredient from the micro-reservoir to the micro-needles (and therefore to the patient, since the micro-needles are inserted in the skin) can exploit the flexibility of the device. For example, it can be expected that the activation of the release of the active ingredient from the micro-reservoir to the micro-needles can take place after a pressure exerted by the patient (in particular by means of a finger) on a wall of the micro-reservoir. Alternatively, the activation of the release of the active ingredient from the micro-reservoir can take place automatically following a variation of the curvature of the support element (in particular from concave to convex) occurring when the device is applied to the patient's skin.

The device according to the present invention can also be arranged for sampling biological fluids. By way of a purely explanatory and non-limiting example, a configuration can be considered wherein the device is equipped with a plurality of micro-needles with through cavities and a micro-reservoir for collecting and/or storing biological fluids (micro-needles and micro-reservoirs being manufactured by photolithography on opposite sides of the support element), the cavities in the micro-needles being in fluid connection with the micro-reservoir through micro-ducts. In this configuration, the physical phenomenon of capillarity can be used so that, once micro-needles passing through the patient's skin have been inserted, the biological fluid (for example blood or interstitial liquid) reaches and fills the micro-reservoir.

From what has been described and/or represented, it is clear how the present invention achieves all the objectives for which it was conceived (in particular, each of the aforementioned objectives from the first objective to the ninth objective) and ensures considerable advantages. For example, the present invention allows a simple and fast manufacture of hollow micro-needles, since a single photolithographic operation is sufficient for obtaining micro-needles with blind or through cavities. The manufacture of micro-needles by photolithography is also appropriate for a large-scale industrial implementation, with very low costs, and it is characterized by the ease in making changes to the geometric characteristics of micro-needles (depending on their future use), given that such variations can be made simply by changing the photolithographic mask and/or by changing the exposure time to energy radiation.

1. Method for the optical activation of the release of an active ingredient by means of a micro-needle (7; 8; 9), said micro-needle (7; 8; 9) being made of a polymeric material added with a photosensitive polymer or with a photosensitive compound, a blind cavity (77) being defined in said micro-needle (7; 8; 9) and containing said active ingredient, comprising the step of:
exposing said micro-needle (7; 8; 9) to a coherent radiation having a predetermined wavelength such to energize said photosensitive polymer or said photosensitive compound, said coherent radiation being preferably in the near infrared field.

2. Method for the thermal activation of the release of an active ingredient by means of a micro-needle (7; 8; 9), said micro-needle (7; 8; 9) being made of a polymeric material added with metal particles, preferably particles of a noble metal, even more preferably gold particles, a blind cavity (77) being defined in said micro-needle (7; 8; 9) and containing said active ingredient, comprising the step of:
exposing said micro-needle (7; 8; 9) to a coherent radiation having a predetermined wavelength such to heat by radiation said metal particles, said coherent radiation being preferably in the near infrared field.

3. Method for obtaining through photolithography at least one micro-needle (7; 8; 9) for the transdermal administration of active molecules and/or the sampling of biological fluids, comprising the step of:
exposing a photo-cross linking polymer in liquid phase to an energy radiation capable of causing the hardening thereof, a photolithographic mask (1; 2) being interposed between the source of said energy radiation and said photo-cross linking polymer, said photolithographic mask (1; 2) being configured in a manner such to generate in said photo-cross linking polymer a peripheral shadow area, a central shadow area and a lighting area confined between said peripheral shadow area and said central shadow area, specifically with the aim of obtaining a hollow micro-needle (7; 8; 9) by means of said photolithography, wherein said photolithographic mask (1; 2) comprises a peripheral region (4; 6) of impermeability to said energy radiation and a central region (3; 5) of impermeability to said energy radiation, said peripheral region (4; 6) being suitable to generate said peripheral shadow area and said central region (3; 5) being suitable to generate said central shadow area, and wherein said peripheral region (4; 6) and said central region (3; 5) are distinct and separate from each other,
wherein, the outer profile (40; 60) of said photolithographic mask (1; 2) being the line that internally delimits said peripheral region (4; 6) and the inner profile (30; 50) of said photolithographic mask (1; 2) being the line that externally delimits said central region (3; 5), said outer profile (40; 60) entirely encloses said inner profile (30; 50), and
wherein the geometric center (C4; C6) of said outer profile (40; 60) is arranged at a predetermined distance (f) with respect to the geometric center (C3; C5) of said inner profile (30; 50), specifically for the purpose to obtain an asymmetric development of said micro-needle (7; 8; 9) during said photolithography.

4. Method according to claim 3, wherein the predetermined distance (f) between the geometric center (C4; C6) of said external profile (40; 60) and the geometric center (C3; C5) of said inner profile (30; 50) is comprised between 10 micrometers and 200 micrometers, preferably between 30 micrometers and 50 micrometers, even more preferably about 40 micrometers.

5. Method for obtaining through photolithography at least one micro-needle (7; 8; 9) for transdermal administration of active molecules and/or for the sampling of biological fluids, comprising the step of:
exposing a photo-cross linking polymer in liquid phase to an energy radiation capable of causing the hardening thereof, a photolithographic mask (1; 2) being interposed between the source of said energy radiation and said photo-cross linking polymer, said photolithographic mask (1; 2) being configured in a manner such to generate in said photo-cross linking polymer a peripheral shadow area, a central shadow area and a lighting area confined between said peripheral shadow area and said central shadow area, specifically with the aim of obtaining a hollow micro-needle (7; 8; 9) by means of said photolithography, further comprising a step between:
interrupting the exposure of said photo-cross linking polymer to said energy radiation before a predetermined duration, specifically with the aim of obtaining a through cavity (88; 99) in said micro-needle (7; 8; 9);
interrupting the exposure of said photo-cross linking polymer to said energy radiation after a predetermined duration, specifically with the aim of obtaining a blind cavity (77) in said micro-needle (7; 8; 9);
setting the power of said source of said energy radiation below a predetermined power, specifically with the aim of obtaining a through cavity (88; 99) in said micro-needle (7; 8; 9);
setting the power of said source of said energy radiation above a predetermined power, specifically with the aim of obtaining a blind cavity (77) in said micro-needle (7; 8; 9).

6. Method for obtaining through photolithography at least one micro-needle (7; 8; 9) for the transdermal administration of active molecules and/or for the sampling of biological fluids, comprising the phase of:
exposing a photo-cross linking polymer in liquid phase to an energy radiation capable of causing the hardening thereof, a photolithographic mask (1; 2) being interposed between the source of said energy radiation and said photo-cross linking polymer, said photolithographic mask (1; 2) being configured in a manner such to generate in said photo-cross linking polymer a peripheral shadow area, a central shadow area and a lighting area confined between said peripheral shadow area and said central shadow area, specifically with the aim of obtaining a hollow micro-needle (7; 8; 9) by means of said photolithography,
wherein the said photo-cross linking polymer is added with a photosensitive polymer or with a photosensitive compound, in particular in order to make the micro-needle (7; 8; 9) suitable for being used to release an active ingredient only under conditions of exposure of the said micro-needle (7; 8; 9) to a coherent radiation of predetermined wavelength and/or
wherein said photo-cross linking polymer is added with metal particles, preferably with particles of a noble metal, even more preferably with gold particles, specifically with the aim of making said micro-needle (7; 8; 9) suitable to be used to release an active ingredient only in conditions of heating by irradiation of said micro-needle (7; 8; 9) and/or
wherein said photo-cross linking polymer is added with an active ingredient.

7. Method for obtaining through photolithography at least one micro-needle (7; 8; 9) for the transdermal administration of active molecules and/or for the sampling of biological fluids, comprising the step of:
exposing a photo-cross linking polymer in liquid phase to an energy radiation capable of causing the hardening thereof, a photolithographic mask (1; 2) being interposed between the source of said energy radiation and said photo-cross linking polymer, said photolithographic mask (1; 2) being configured in a manner such to generate in said photo-cross linking polymer a peripheral shadow area, a central shadow area and a lighting area confined between said peripheral shadow area and said central shadow area, specifically with the aim of obtaining a hollow micro-needle (7; 8; 9) by means of said photolithography,
wherein the molecular weight of said photo-cross linking polymer is modular so as to confer to said micro-needle (7; 8; 9) morphological characteristics such to adjust the speed of release of the molecules of an active ingredient through said micro-needle (7; 8; 9) and/or
wherein the wettability of said photo-cross linking polymer is modular so as to confer to said micro-needle (7; 8; 9) surface chemical characteristics and/or according to the hydrophobic and/or hydrophilic of an active ingredient to be released through said micro-needle (7; 8; 9).

8. Method according to claim 3, further comprising the step of:
removing from said micro-needle (7; 8; 9) the non-hardened photo-cross linking polymer by washing said micro-needle (7; 8; 9), in particular in deionised water.

9. Method according to claim 3, wherein said micro-needle (7; 8; 9) is obtained simultaneously with at least one further micro-needle, optionally simultaneously with a plurality of micro-needles positioned based on a predetermined regular and/or orderly arrangement.

10. Method according to claim 3, wherein said micro-needle (7; 8; 9) is generated on a surface of a support element, said surface of said support element having an opening at the position intended for said micro-needle (7; 8; 9),
optionally wherein said photo-cross linking polymer is contained in a recipient, preferably made of silicone, and said support element is placed on said recipient so as to be at direct contact with said photo-cross linking polymer.

11. Micro-needle (7; 8; 9) for the transdermal administration of active molecules and/or for sampling biological fluids, said micro-needle (7; 8; 9) being made of polymeric material, wherein a cavity is defined in said micro-needle (7; 8; 9),
wherein said polymeric material is added with a photosensitive polymer or with a photosensitive compound and/or
wherein said polymeric material is added with metal particles, preferably with particles of a noble metal, even more preferably with gold particles and/or
wherein said polymeric material is added with an active ingredient.

12. Micro-needle (7; 8; 9) according to claim 11, wherein said micro-needle (7; 8; 9) is straight truncated-cone shaped or a regular truncated-pyramid shaped and wherein said cavity is a through cavity (88; 99) or wherein said micro-needle (7; 8; 9) is straight cone-shaped or regular pyramid-shaped and wherein said cavity is a blind cavity (77) or wherein said micro-needle (7; 8; 9) is oblique truncated cone-shaped or irregular truncated-pyramid shaped and wherein said cavity is a through cavity (88; 99).

13. Micro-needle (7; 8; 9) according to claim 11, wherein the height (h7; h8; h9) of said micro-needle (7; 8; 9) is comprised between 200 micrometres and 2000 micrometres, preferably between 900 micrometres and 1300 micrometres, even more preferably about 1100 micrometres, and/or wherein the base (70; 80; 90) of said micro-needle (7; 8; 9) has a diameter (r70; r80; r90) or diagonal with extension comprised between 100 micrometres and 900 micrometres, preferably between 300 micrometres and 700 micrometres, even more preferably about 500 micrometres, and/or wherein the thickness (k7; k8; k91, k92) of the walls of said micro-needle (7; 8; 9) is comprised between 10 micrometres and 200 micrometres, preferably between 60 micrometres and 140 micrometres, even more preferably about 100 micrometres.

14. Device for the transdermal administration of active molecules and/or for sampling biological fluids, comprising at least one micro-needle (7; 8; 9) and a support element, said micro-needle (7; 8; 9) being made of material polymeric, a cavity being defined in said micro-needle (7; 8; 9), said at least one micro-needle (7; 8; 9) extending from a surface of said support element moving away from said support element,
wherein said surface of said support element is flexible.

15. Device according to claim 14, wherein said surface of said support element has an opening at said micro-needle (7; 8; 9), optionally the shape of said at least one opening being substantially identical to the shape of the base of said at least one micro-needle (7; 8; 9) and/or the diameter or the diagonal of said at least one opening having an extension substantially identical to the extension of the diameter (r70; r80; r90) or of the diagonal of the base (70; 80; 90) of said at least one micro-needle (7; 8; 9).

16. Device according to claim 14, comprising a plurality of micro-needles, each micro-needle (7; 8; 9) of said plurality being made of polymeric material, a cavity being defined in each micro-needle (7; 8; 9) of said plurality, said micro-needles extending from said surface of said support element moving away from said support element, said micro-needles being positioned on said surface of said support element depending on a predetermined regular and/or orderly arrangement.

17. Device according to claim 14, wherein an active ingredient is contained in the cavity of said micro-needle (7; 8; 9) and/or in the cavities of said micro-needles.

18. Device according to claim 14, further comprising at least one micro-fluidic circuit and/or at least one micro-duct and/or at least one micro-reservoir in fluid communication with the cavity of said micro-needle (7; 8; 9) and/or with the cavities of said micro-needles.
